(19) United States
(12) Patent Application Publication
(10) Pub. No.: US 2021/0060105 A1
(43) Pub. Date: Mar. 4, 2021

(54) GINKGO BILOBA EXTRACT COMPOSITE FORMULATION AND USE OF THE SAME

(71) Applicants: Yong Luke ZHANG, Boulder, CO (US); A Hou ZHANG, Boulder, CO (US)

(72) Inventors: Yong Luke ZHANG, Boulder, CO (US); A Hou ZHANG, Boulder, CO (US)

(21) Appl. No.: 16/553,188

(22) Filed: Aug. 28, 2019

(51) Int. Cl. A61K 36/16; A61K 36/534; A61K 31/122; A61K 31/343; A61K 31/192; A61K 31/25; A61K 36/258; A61K 36/481

(52) U.S. Cl. CPC A61K 36/16; A61K 36/534; A61K 31/122; A61K 36/481; A61K 31/192; A61K 31/25; A61K 36/258; A61K 31/343

(57) ABSTRACT

The present invention discloses a Ginkgo Biloba extract composite formulation and the use thereof, comprising: a Ginkgo Biloba extract, a Salvia miltiorrhiza extract, astaxanthin, Astragalus, Coenzyme Q10 and American Ginseng; the Ginkgo Biloba extract comprises Ginkgo flavonoids and Ginkgolides; the Salvia miltiorrhiza extract comprises tanshinone II A, Danshensu and Salvianolic acid. The invention combines the Ginkgo Biloba extract, the Salvia miltiorrhiza extract, astaxanthin, Astragalus, Coenzyme Q10 and American Ginseng for comprehensive preparation; effectively conditions the disordered body through comprehensive nourishing and conditioning, thus truly solving the “fatigue” and supplementing vitality, and recuperating the body to a healthy state. The Ginkgo Biloba extract composite formulation of the invention can be widely used in patients with cardiovascular and cerebrovascular diseases, “three high symptoms”, and has a variety of functions such as anti-cancer, anti-fatigue, anti-hypoxia, anti-radiation, anti-aging and enhancing immunity.

### TECHNICAL FIELD

The invention relates to a health-care food, in particular to a Ginkgo Biloba extract composite formulation and use which can prevent and treat cardiovascular and cerebrovascular diseases, “three high symptoms” and enhance immunity based on the homology of medicines and foods.

### BACKGROUND OF THE INVENTION

Nowadays, the global aging phenomenon is becoming more and more serious. The function of multiple organs in the middle-aged and elderly people is naturally declining, the immunity is declining, and the “three high symptoms”—high blood pressure, high blood fat, high blood clots, seriously threaten the health and life safety of the middle-aged and elderly people, and the resulting cardiovascular and cerebrovascular diseases and renal vascular diseases are the main causes of death and disability.

At present, the “three high symptoms” are effectively treated by using chemical medicines, but the liver and kidney functions of the middle-aged and elderly people are degrading, and they often have many diseases simultaneously. If multiple medicines are used together, it may cause side effects such as drug-induced diseases and iatrogenic death. The fatalities due to the above-mentioned reasons in the developed Western countries are reported to account for 18% of the total number of hospital deaths, which is much higher than this in developing countries.

Using Chinese medicines to treat the “three high symptoms”, the diseases in the middle-aged and elderly people caused by the decline of immunity can obviously avoid the side effects caused by treatment with chemical medicines. Furthermore, the homologies of medicines and foods are the essence of traditional Chinese medicines, so it is necessary to develop a health-care food to treat the “three high symptoms”, enhance immunity and anti-aging. At present, there are lots of products related to middle-aged and elderly health care, but they all have the characteristics of single effect, inconvenient use, not easy to absorb, slow effect, etc., and the price is very high, and it is difficult to popularize.

### SUMMARY OF THE INVENTION

In view of the deficiencies in the above problems, the present invention provides a Ginkgo Biloba extract composite formulation and use of the same.

The invention discloses a Ginkgo Biloba extract composite formulation, comprising: a Ginkgo Biloba extract, a Salvia miltiorrhiza extract, astaxanthin, Astragalus, Coenzyme Q10 and American Ginseng.

the Ginkgo Biloba extract comprises Ginkgo flavonoids and Ginkgolides, and the Salvia miltiorrhiza extract comprises tanshinone II A, Danshensu and Salvianolic acid.

As a further improvement of the present invention, based on the total weight, the Ginkgo Biloba extract accounts for 1-60 parts, the Salvia miltiorrhiza extract accounts for 1-60 parts, the astaxanthin accounts for 1-50 parts, the Astragalus accounts for 1-20 parts, the Coenzyme Q10 accounts for 1-30 parts, and the American Ginseng accounts for 1-25 parts.

As a further improvement of the present invention, based on the total weight, the Ginkgo Biloba extract accounts for 35-45 parts, the Salvia miltiorrhiza extract accounts for 18-26 parts, the astaxanthin accounts for 15-18 parts, the Astragalus accounts for 12-15 parts, the Coenzyme Q10 accounts for 12-15 parts, and the American Ginseng accounts for 9-10 parts.

As a further improvement of the present invention, the weight percentage of the Ginkgo flavonoids is 1-60%, and the weight percentage of the Ginkgolides is 0.5-10%.

As a further improvement of the present invention, the weight percentage of the Ginkgo flavonoids is 20-40%, and the weight percentage of the Ginkgolides is 4-10%.

As a further improvement of the present invention, the weight percentage of the tanshinone II A is 1-60%, the weight percentage of the Danshensu is 1-60%, and the weight percentage of Salvianolic acid is 1-60%.

As a further improvement of the present invention, the weight percentage of the tanshinone II A is 20-40%, the weight percentage of the Danshensu is 20-40%, and the weight percentage of Salvianolic acid is 20-40%.

The invention also discloses a use of the Ginkgo Biloba extract composite formulation, comprising: adding an excipient to the Ginkgo Biloba extract, Salvia miltiorrhiza extract, astaxanthin, Astragalus, Coenzyme Q10, and American Ginseng, and processing the mixture to prepare any one of a pill, a soft capsule, a hard capsule, a granule, a tablet, and an oral solution.

Compared with the prior art, the beneficial effects of the present invention are:

the Ginkgo Biloba extract composite formulation of the invention is based on the expansion effect of Ginkgo flavonoids and Ginkgolides on arterial blood vessels; on the protective effects of tanshinone IIA, Danshensu and Salvianolic acid on myocardial ischemia and the functions of promoting blood circulation and removing blood stasis thereof; and on the function of supplementing vitality of Astragalus. By means of comprehensively formulating the Ginkgo Biloba extract, the Salvia miltiorrhiza extract, astaxanthin, Astragalus, Coenzyme Q10 and American Ginseng and comprehensively nourishing and conditioning, the blood viscosity can be reduced and the blood pressure can be lowered while the blood vessels are expanded, etc. On such a basis, the disordered body can be effectively nourished, thus truly solving the “fatigue”, supplementing the vitality, and restoring the body to a healthy state. The Ginkgo Biloba extract composite formulation of the invention can be widely used in patients with cardiovascular and cerebrovascular diseases, and with “three high symptoms”. Moreover, it has multiple functions such as anti-cancer, anti-fatigue, anti-hypoxia, anti-radiation, anti-aging, and enhanced immunity, etc.

In the present invention, the Ginkgo Biloba extract composite formulation can be processed into any one of a pill, a soft capsule, a hard capsule, a granule, a tablet or an oral solution, and has the advantages of convenient use, rapid absorption and significant curative effects, easy to produce, low cost and other characteristics, and also has a dual value of foods and medicines, to meet the health needs of consumers, which is the latest variety of similar health-care foods.

### DETAILED DESCRIPTION OF THE EMBODIMENT

The embodiments of the present invention will be described clearly and completely in the following description of the embodiments of the present invention. It is obvious that the described embodiments are a part of the embodiments of the present invention, and not all of them. Based on the embodiments of the present invention, all other embodiments obtained by a person of ordinary skills in the art based on the embodiments of the present invention without creative efforts are within the scope of the present invention.

The present invention is further described in detail below.

The invention provides a Ginkgo Biloba extract composite formulation, comprising: a Ginkgo Biloba extract, a Salvia miltiorrhiza extract, astaxanthin, Astragalus, Coenzyme Q10 and American Ginseng.

The Ginkgo Biloba extract composite formulation of the present invention may further comprise a medicinal excipient in addition to the above-mentioned main ingredients, and the medicinal excipient includes one or more of a diluent, an absorbent, a wetting agent and the like.

According to the principle of traditional Chinese medicine, the invention uses a Ginkgo Biloba extract, a Salvia miltiorrhiza extract, astaxanthin, Astragalus, Coenzyme Q10 and American Ginseng as main components to prepare a Ginkgo Biloba extract composite formulation; which can enhance human's immunity and improve their health, and is more conducive to absorption and the curative effect thereof is more obvious. Due to the complexity and a great deal of variability in the disease courses and natures, it is often interlaced with cold syndrome and heat syndrome, both deficiency syndrome and excess syndrome are present, and there are several diseases occurred in one patient at the same time. It is difficult to cover all these aspects with a single health-care product. Therefore, it is necessary to properly cooperate several health-care products and use synergy or antagonism between them, thus improving the efficacy or reducing adverse reactions to adapt to the treatment of complex diseases and daily health care.

In order to play the synergy among the Ginkgo Biloba extract, the Salvia miltiorrhiza extract, astaxanthin, Astragalus, Coenzyme Q10 and American Ginseng better, so as to obtain the optimum effects in the prevention and treatment of cardiovascular and cerebrovascular diseases and “three high symptoms”, and in the improvement of immunity, according to the invention, based on the total weight, the Ginkgo Biloba extract accounts for 1-60 parts, the Salvia miltiorrhiza extract accounts for 1-60 parts, astaxanthin accounts for 1-50 parts, Astragalus 1-20 parts, and Coenzyme Q10 accounts for 1-30 parts, American Ginseng accounts for 1-25 parts; preferably, 35-45 parts of the Ginkgo Biloba extract, 18-26 parts of the Salvia miltiorrhiza extract, 15-18 parts of astaxanthin, 12-15 parts of Astragalus, 12-15 parts of Coenzyme Q10, and 9-10 parts of American Ginseng.

The Ginkgo Biloba extract comprises a lot of components. The Ginkgo Biloba extract selected by the present invention comprises Ginkgo flavonoids and Ginkgolides, and the weight percentage of Ginkgo flavonoids is 1-60%, and the weight percentage of Ginkgolides is 0.5-10%; preferably, the weight percentage of Ginkgo flavonoids is 20-40%, and the weight percentage of Ginkgolides is 4-10%.

The Salvia miltiorrhiza extract comprises a lot of components. The Salvia miltiorrhiza extract in the invention comprises tanshinone II A, Danshensu and Salvianolic acid, and the weight percentage of tanshinone II A is 1-60%, and the weight percentage of Danshensu is 1-60%, the weight percentage of Salvianolic acid is 1-60%; preferably, the weight percentage of tanshinone IIA is 20-40%, the weight percentage of Danshensu is 20-40%, the weight percentage of Salvianolic acid is 20-40%.

The invention also provides a use of the Ginkgo Biloba extract composite formulation, comprising: adding an excipient to the Ginkgo Biloba extract, Salvia miltiorrhiza extract, astaxanthin, Astragalus, Coenzyme Q10 and American Ginseng, and processing the mixture to prepare any one of a pill, a soft capsule, a hard capsule, a granule, a tablet and an oral solution, so that the health needs of different consumers can be met.

The efficacy of each active component in the Ginkgo Biloba extract composite formulation of the present invention is as follows:

Ginkgo Biloba: Ginkgo Biloba, also known as Ginkgo, commonly known as Gongsunshu, firstly appeared in the Carboniferous Age 345 million years ago, and became a unique tree species in China after the Quaternary continental glaciers, native to China, then it was introduced to Europe around 1710. It is referred to “living fossils”, “the panda in the plant world”. A large number of domestic and abroad studies have shown that the chemical composition of Ginkgo Biloba leaves is very complicated. The main biological active ingredients are flavonoids, azlactones, polyprenols and polysaccharides, and also contain organic acids, alkylphenolic acids, amino acids, terpenoids, trace elements, etc. The efficacy thereof is as follows: (1) lowering the level of cholesterol in the blood of the human body, preventing arteriosclerosis, and significantly improving the physical strength, rapid heartbeat, chest pain, dizziness, etc. after slight activity in middle-aged and elderly people; (2) reducing the blood pressure by increasing the vascular permeability and elasticity, thus having better antihypertensive effect; (3) eliminating deposition components on the blood vessel wall, improving blood rheology, improving the deformability of the red blood cell, reducing blood viscosity, and making the blood flow smooth, thus preventing and treating cerebral hemorrhage and cerebral infarction, and also having certain curative effects on senile dementia caused by arteriosclerosis; (4) the Ginkgo Biloba formulation and hypoglycemic agent have better curative effect on diabetes, and can be used as auxiliary medicine for diabetes; (5) significantly reducing symptoms such as menstrual abdominal pain and pain in back and waist; (6) using for the treatment of bronchial asthma, and also having a good effect; (7) reducing lipid peroxidation levels, reducing freckles, moisturizing skins; (8) constraining the lung, suppressing cough, activating blood circulation to dissipate blood stasis, relieving pain, using for lung vacuity cough and panting, coronary heart diseases, angina, hyperlipemia, anticoagulate, having a certain probability of improving memory. The Ginkgo flavonoids and Ginkgolides in the Ginkgo Biloba extract have an expanding effect on cardiovascular and cerebrovascular blood vessels, and are important for the prevention and treatment of cardiovascular and cerebrovascular diseases. Ginkgo leaves can be used to treat oppression in the chest and cardiodynia, to improve blood circulation in the heart and brain, lower cholesterol and improve symptoms such as high blood pressure. The roots of Ginkgo trees can be used to nourish and strengthen the weak body. Ginkgo Biloba has many miraculous effects. Under the verification of scientific tests, the most notable effects include: promoting blood circulation, preventing cardiovascular and cerebrovascular diseases; enhancing memory, preventing Alzheimer's disease; and anti-oxidation. Whether in Germany, the United States, Japan or the mainland of China, the research on Ginkgo is still coming out, and Ginkgo leaves have become a healthy food popular in many countries.

Ginkgo flavonoids have pharmacological effects such as expanding blood vessels, inhibiting platelet activating factor, anti-oxidation, regulating blood lipids and anti-tumor. They have special curative effects on treating angina and myocardial infarction, etc. Ginkgolides are also scavengers of peroxidic free radicals and can eliminate free radicals that are toxic to endothelial cells in the cardiovascular and cerebrovascular blood vessels, thus having health-care effects such as anti-aging and anti-cancer.

Ginkgolides mainly include diterpene lactones and sesquiterpene lactones. There are five kinds of diterpene lactones, namely, Ginkgolides A, B, C, M and J, abbreviated as GA, GB, GC, GM, GJ; one kind of sesquiterpene lactone, namely bilobalide, referred to as BB. Ginkgolides have a specific platelet activating factor (PAF) activity and is a natural PAF antagonist. They can treat asthma, lung allergy, heart failure and other diseases. Among them, GB has the highest activity, and bilobalide is a neuropsychiatric drug that is resistant to the aging of nerve endings and is a true anti-aging substance.

Salvia miltiorrhiza: “One medicinal of Salvia miltiorrhiza, its effect corresponds to four kinds of medicinals” (Rehmannia, Chinese Angelica, Radix Paeoniae Alba, Rhizoma Ligustici), it is recognized in traditional Chinese medicine that: Salvia miltiorrhiza integrates nourishing blood, promoting blood circulation, dissipating blood stasis, relieving pain, and engendering new blood together, with remarkable efficacy and moderate flavor and nature, clearing heart and trouble, both supplementing and dispelling, without any toxic side effects. The effect of Salvia miltiorrhiza on the central nervous system is mainly the inhibition of the cerebral cortex, which produces a sedative effect. The Salvia miltiorrhiza formulation has certain anti-tumor effects in vitro and in vivo. Clinically, Salvia miltiorrhiza and radiation therapy are combined for tumor patients. Various extracts of Salvia miltiorrhiza have obvious antibacterial and anti-inflammatory effects. In conclusion, Salvia miltiorrhiza has anti-oxidation, protective effects on brain tissue ischemia and reperfusion injury, enhances hypoxia tolerance and improves renal function.

Astaxanthin: astaxanthin is a kind of carotenoid and is a strong natural antioxidant. Like other carotenoids, astaxanthin is a fat-soluble and water-soluble pigment found in marine organisms such as shrimps, crabs, squids, and algaes. Astaxanthin has a strong antioxidant ability of 550 times that of vitamin E and 10 times that of β-carotene. Astaxanthin is one of the most effective natural antioxidants. Its main function is to scavenge free radicals and improve the body's anti-aging ability. Natural astaxanthin can penetrate the three major human bathers of blood-brain barrier, blood-pancreas barrier and blood-testis barrier, and is the only carotenoid that can penetrate the blood-brain barrier, so it is the only kind of carotenoid capable of acting on brain cells and eyeball retina. Astaxanthin can significantly inhibit chemical-induced initial canceration, and has anti-proliferative effects and immune function enhancement on epithelial cells exposed to carcinogens, such as inhibit the developing of bladder cancer, oral cancer, colon cancer, gastric cancer and breast cancer. Astaxanthin can also reduce the carcinogenicity of aflatoxins and works well to reduce the amount and volume of aflatoxin-induced liver tumor cells. Studies have shown that astaxanthin has an effect of significantly increasing HDL and reducing LDL in vivo, in which HDL can be increased from the original 49.7±3.6 mg/dL to 66.5±5.1 mg/dL, so it is speculated that astaxanthin can reduce the oxidation of apolipoprotein, and can be used to prevent arteriosclerosis, coronary heart diseases and ischemic brain damage.

Coenzyme Q10: Coenzyme Q10 plays an important role in proton translocating and electron transporting in the human respiration chain. It acts as a cellular metabolism and cellular respiration activator. It is also an important antioxidant and specific immunopotentiator, can promote oxidative phosphorylation, and protect the integrity of the cell membrane structure. Coenzyme Q10 is essential for the human body like the 6 major nutrients, and it can be used as an indicator of human health. The experimental data showed that people with cardiovascular and cerebrovascular diseases, chemoradiotherapy patients, AIDS, hepatitis, cancer patients, and elderly weak people with high blood pressure, had lower Coenzyme Q10 in plasma, tissues and organs than healthy people. Scientists around the world have done a lot of research on the mechanism of Coenzyme Q10 improving heart function. It is found that Coenzyme Q10 can reduce low-density lipoprotein, prevent low-density lipoprotein from penetrating endothelial cells, deep into the endothelial cell gap, and reduce the formation of lipids on the inner wall of arteries. It can also increase the content of high-density lipoprotein called “vascular scavenger”, remove the garbage, toxins and plaque formed by the inner wall of blood vessels, and two-way regulate blood lipids and prevent atherosclerosis.

American Ginseng: American Ginseng is natured cool, and tastes sweet and slightly bitter. In the protection of the cardiovascular system: often taking American Ginseng can resist arrhythmia, anti-myocardial ischemia, anti-myocardial oxidation, strengthen myocardial contractility. Coronary heart disease patients with symptoms of qi and yin deficiency, palpitation and shortness of breath can take long-term use of American Ginseng, the effect is significant. American Ginseng is also effective in regulating blood pressure, which can effectively reduce temporary and persistent blood pressure, and contribute to the recovery of hypertension, arrhythmia, coronary heart diseases, acute myocardial infarction, and cerebral thrombosis. In terms of improving immunity: American Ginseng, used as the preferred health-care medicine for invigorating qi, can promote serum protein synthesis, bone marrow protein synthesis, organ protein synthesis, etc., improve the body's immunity, inhibit cancer cell growth, and effectively resist cancers. Long-taking American Ginseng can reduce blood coagulation, inhibit platelet aggregation, resist atherosclerosis and promote red blood cell growth, and increase hemoglobin. American Ginseng can lower blood sugar, regulate insulin secretion, promote glucose metabolism and fat metabolism, and has certain auxiliary effects on the treatment of diabetes.

Astragalus: Astragalus is natured warm and tastes sweet, and it is in the lung and spleen channels, and is a very important qi-invigorating medicine. Not only can it supplement the body's qi, but also is good at supplementing the qi of the surficial tissues, especially for the patients with fatigue, lack of energy, apathetic and loss of appetite caused by spleen deficiency. It has the following effects:

1. benefiting vital energy and securing the exterior: Astragalus is natured more warm, tastes sweet, and is in the spleen channels, is an important medicine vital energy for supplementing the vital energy of the spleen, can treat deficiency and weakness in the vital energy of the spleen, fatigue, deficiency in energy, decreased food intake and loose stool; 2. regulating blood pressure: its antihypertensive components are γ-aminobutyric acid and Astragaloside IV, and have a two-way regulation of blood pressure, which can significantly reduce the resistance of cerebral vessels, peripheral blood vessels, coronary arteries, and mesenteric arteries, and have a certain expansion effect on blood vessels in these parts, but have a contractile effect on renal blood vessels; 3. increasing the body's immune function: capable of significantly increasing the total number of white blood cells in the blood, promoting the phagocytic function and bactericidal function of neutrophils and macrophages, enhancing the body's immunity; 4. cardiotonic function: increasing the amplitude of heart contraction, increasing the output, more obvious effects on toxication or heart failure. Moreover, Astragalus polysaccharide can effectively fight acute myocardial ischemia caused by pituitrin; it can be taken directly soaked in water daily. However, their medicinal value is mainly taken, patients who have excess syndromes or yin deficiency should avoid eating, other patients instead taking Astragalus, such as cold, acute gastroenteritis, abdominal fullness are not suitable for taking Astragalus.

### Example 1

The invention provides a Ginkgo Biloba extract composite formulation, which comprises, by weight:

60 parts of the Ginkgo Biloba extract, in which the weight percentage of Gingko flavonoids is 60% and the weight percentage of Ginkgolides is 10%;

1 part of the Salvia miltiorrhiza extract, in which the weight percentage of tanshinone II A is 39%, the weight percentage of Danshensu is 60%, and the weight percentage of Salvianolic acid is 1%;

1 part of astaxanthin;

20 parts of Astragalus;

30 parts of Coenzyme Q10;

25 parts of American Ginseng.

In the present invention, an excipient is added to the above components, and processed according to a conventional health-care food processing method to prepare a pill.

### Example 2

The invention provides a Ginkgo Biloba extract composite formulation, which comprises, by weight:

1 part of the Ginkgo Biloba extract, in which the weight percentage of Gingko flavonoids is 1% and the weight percentage of Ginkgolides is 0.5%;

60 parts of the Salvia miltiorrhiza extract, in which the weight percentage of tanshinone II A is 60%, the weight percentage of Danshensu is 1%, and the weight percentage of Salvianolic acid is 39%;

50 parts of astaxanthin;

1 part of Astragalus;

1 part of Coenzyme Q10;

1 part of American Ginseng.

In the present invention, an excipient is added to the above components, and processed according to a conventional health-care food processing method to prepare a capsule.

### Example 3

The invention provides a Ginkgo Biloba extract composite formulation, which comprises, by weight:

part of the Ginkgo Biloba extract, in which the weight percentage of Gingko flavonoids is 1% and the weight percentage of Ginkgolides is 10%;

60 parts of the Salvia miltiorrhiza extract, in which the weight percentage of tanshinone II A is 1%, the weight percentage of Danshensu is 39%, and the weight percentage of Salvianolic acid is 60%;

50 parts of astaxanthin;

1 part of Astragalus;

1 part of Coenzyme Q10;

1 part of American Ginseng.

In the present invention, an excipient is added to the above components, and processed according to a conventional health-care food processing method to prepare a granule.

### Example 4

The invention provides a Ginkgo Biloba extract composite formulation, which comprises, by weight:

30 parts of the Ginkgo Biloba extract, in which the weight percentage of Gingko flavonoids is 20% and the weight percentage of Ginkgolides is 10%, or the weight percentage of Gingko flavonoids is 40% and the weight percentage of Ginkgolides is 4%;

30 parts of the Salvia miltiorrhiza extract, in which the weight percentage of tanshinone II A is 20%, the weight percentage of Danshensu is 40% and the weight percentage of Salvianolic acid is 40%, or the weight percentage of tanshinone II A is 40%, the weight percentage of Danshensu is 20% and the weight percentage of Salvianolic acid is 40%, or the weight percentage of tanshinone II A is 40%, the weight percentage of Danshensu is 40% and the weight percentage of Salvianolic acid is 20%; 20 parts of astaxanthin;

20 parts of Astragalus;

20 parts of Coenzyme Q10;

10 parts of American Ginseng.

In the present invention, an excipient is added to the above components, and processed according to a conventional health-care food processing method to prepare a tablet.

### Example 5

The invention provides a Ginkgo Biloba extract composite formulation, exemplified by that: in the Ginkgo Biloba extract, the weight percentage of Gingko flavonoids is 20-40% and the weight percentage of Ginkgolides is 4-10%;

in the Salvia miltiorrhiza extract, the weight percentage of tanshinone II A is 20-40%, the weight percentage of Danshensu is 20-40%, and the weight percentage of Salvianolic acid is 20-40%;

the composite formulation comprises, by weight:

35 parts of the Ginkgo Biloba extract;

25 parts of the Salvia miltiorrhiza extract;

18 parts of astaxanthin;

12 part of Astragalus;

12 part of Coenzyme Q10;

10 part of American Ginseng.

In the present invention, an excipient is added to the above components, and processed according to a conventional health-care food processing method to prepare a soft capsule.

### Example 6

The invention provides a Ginkgo Biloba extract composite formulation, exemplified by that: in the Ginkgo Biloba extract, the weight percentage of Gingko flavonoids is 20-40% and the weight percentage of Ginkgolides is 4-10%;

in the Salvia miltiorrhiza extract, the weight percentage of tanshinone II A is 20-40%, the weight percentage of Danshensu is 20-40%, and the weight percentage of Salvianolic acid is 20-40%;

the composite formulation comprises, by weight:

38 parts of the Ginkgo Biloba extract;

26 parts of the Salvia miltiorrhiza extract;

15 parts of astaxanthin;

12 part of Astragalus;

12 part of Coenzyme Q10;

9 part of American Ginseng.

In the present invention, an excipient is added to the above components, and processed according to a conventional health-care food processing method to prepare a tablet.

### Example 7

The invention provides a Ginkgo Biloba extract composite formulation, exemplified by that: in the Ginkgo Biloba extract, the weight percentage of Gingko flavonoids is 20-40% and the weight percentage of Ginkgolides is 4-10%;
in the Salvia miltiorrhiza extract, the weight percentage of tanshinone II A is 20-40%, the weight percentage of Danshensu is 20-40%, and the weight percentage of Salvianolic acid is 20-40%;

the composite formulation comprises, by weight:

40 parts of the Ginkgo Biloba extract;

20 parts of the Salvia miltiorrhiza extract;

15 parts of astaxanthin;

15 part of Astragalus;

15 part of Coenzyme Q10;

10 part of American Ginseng.

In the present invention, an excipient is added to the above components, and processed according to a conventional health-care food processing method to prepare a pill.

### Example 8

The invention provides a Ginkgo Biloba extract composite formulation, exemplified by that: in the Ginkgo Biloba extract, the weight percentage of Gingko flavonoids is 20-40% and the weight percentage of Ginkgolides is 4-10%;

in the Salvia miltiorrhiza extract, the weight percentage of tanshinone II A is 20-40%, the weight percentage of Danshensu is 20-40%, and the weight percentage of Salvianolic acid is 20-40%;

the composite formulation comprises, by weight:

45 parts of the Ginkgo Biloba extract;

18 parts of the Salvia miltiorrhiza extract;

15 parts of astaxanthin;

13 part of Astragalus;

13 part of Coenzyme Q10; 9 part of American Ginseng.

In the present invention, an excipient is added to the above components, and processed according to a conventional health-care food processing method to prepare a granule.

Experiments:

The test groups are: intaking the soft capsule of Example 5;

Control group 1: intaking the Ginkgo Biloba extract soft capsule, the active ingredients in Ginkgo Biloba extract soft capsule are Ginkgo flavonoids and Ginkgolides, and the active ingredient contents of Ginkgo flavonoids and Ginkgolides are equivalent to those of the soft capsule of Example 5.

Control group 2: intaking Salvia miltiorrhiza extract soft capsule, the active ingredients in the Salvia miltiorrhiza extract soft capsule are tanshinone IIA, Danshensu and Salvianolic acid, the active ingredient contents of tanshinone IIA, Danshensu and Salvianolic acid are equivalent to those of the soft capsule of Example 5.

A large number of people aged 50 to 70 years old are selected for blood pressure and blood lipid testing. The test criteria are:

the normal blood pressure is 80-120 mmHg;

the normal blood lipid range is: (1) total cholesterol≤200 mg/di or low-density lipoprotein cholesterol≤120 mg/di, (2) serum triglycerides≤150 mg/di, (3) high-density lipoprotein cholesterol≥35 mg/di. If any one of the total cholesterol, low-density lipoprotein cholesterol, serum triglyceride or high-density lipoprotein cholesterol is higher than normal blood lipid value, it is considered as high blood lipid.

Based on the above-mentioned physical test standards, three groups of people are selected, namely, the first group was: 300 members with hypertension and hyperlipidemia; the second group was: 300 members with hypertension and normal blood lipids; and the third group was: 300 members with normal blood pressure and high blood lipids.

The first group, the second group and the third group are respectively divided into three subgroups, and the three subgroups divided from each group consumed the soft capsule of Example 5 (test group with 100 persons) and Ginkgo Biloba extract soft capsule (control group 1 with 100 persons) and Salvia miltiorrhiza extract soft capsules (control group 2 with 100 persons), respectively; three times a day, eight capsules for each time (see example 5 for the composition of the soft capsule, 50 mg per capsule); each batch of subjects are measured on the blood pressure and blood lipids respectively after 1 month, 3 months or 6 months, and the number of those whose blood pressure and blood lipids returned to normal among the tested 100 people was counted. The counted results are shown in Table 1.

TABLE 1

| The | | | | | | | | | |
|---|---|---|---|---|---|---|---|---|---|
| number of | | | | | | | | | |
| the subjects | | | | | | | | | |
| with blood | | | | | | | | | |
| pressure | The group with high | | | The group with high | | | The group with normal | | |
| and blood | blood pressure and | | | blood pressure and | | | blood pressure and high | | |
| lipids | high blood lipids | | | normal blood lipids | | | blood lipids | | |
| returning | Tested | Control | Control | Tested | Control | Control | Tested | Control | Control |
| to normal | group | group 1 | group 2 | group | group 1 | group 2 | group | group 1 | group 2 |
| After 1 | 19 | 2 | 5 | 25 | 6 | 7 | 23 | 4 | 9 |
| month | | | | | | | | | |
| After 3 | 42 | 10 | 19 | 56 | 18 | 21 | 53 | 15 | 29 |
| months | | | | | | | | | |
| After 6 | 89 | 24 | 28 | 92 | 32 | 35 | 92 | 28 | 42 |
| months | | | | | | | | | |

It can be seen from the above test that the test results of the present invention clearly show that the present invention has the ideal effects of enhancing immunity, treating three high syndromes, regulating blood lipids, improving health and health care.

The above are only the preferred embodiments of the present invention, and are not intended to limit the present invention, and various modifications and changes can be made to the present invention for the terms of the skilled person in the art. Any modifications, equivalent substitutions, improvements, etc. made within the spirit and scope of the present invention are intended to be included within the scope of the present invention.

1. A Ginkgo Biloba extract composite formulation, characterized in that, comprising: a Ginkgo Biloba extract, a Salvia miltiorrhiza extract, astaxanthin, Astragalus, Coenzyme Q10 and American Ginseng;
the Ginkgo Biloba extract comprises Ginkgo flavonoids and Ginkgolides, and the Salvia miltiorrhiza extract comprises tanshinone II A, Danshensu and Salvianolic acid.

2. The Ginkgo Biloba extract composite formulation according to claim 1, wherein based on the total weight, the Ginkgo Biloba extract accounts for 1-60 parts, the Salvia miltiorrhiza extract accounts for 1-60 parts, the astaxanthin accounts for 1-50 parts, the Astragalus accounts for 1-20 parts, the Coenzyme Q10 accounts for 1-30 parts, and the American Ginseng accounts for 1-25 parts.

3. The Ginkgo Biloba extract composite formulation according to claim 2, wherein based on the total weight, the Ginkgo Biloba extract accounts for 35-45 parts, the Salvia miltiorrhiza extract accounts for 18-26 parts, the astaxanthin accounts for 15-18 parts, the Astragalus accounts for 12-15 parts, the Coenzyme Q10 accounts for 12-15 parts, and the American Ginseng accounts for 9-10 parts.

4. The Ginkgo Biloba extract composite formulation according to claim 1, wherein the weight percentage of the Ginkgo flavonoids is 1-60%, and the weight percentage of the Ginkgolides is 0.5-10%.

5. The Ginkgo Biloba extract composite formulation according to claim 4, wherein the weight percentage of the Ginkgo flavonoids is 20-40%, and the weight percentage of the Ginkgolides is 4-10%.

6. The Ginkgo Biloba extract composite formulation according to claim 1, wherein the weight percentage of the tanshinone II A is 1-60%, the weight percentage of the Danshensu is 1-60%, and the weight percentage of Salvianolic acid is 1-60%.

7. The Ginkgo Biloba extract composite formulation according to claim 6, wherein the weight percentage of the tanshinone II A is 20-40%, the weight percentage of the Danshensu is 20-40%, and the weight percentage of Salvianolic acid is 20-40%.

8. A use of the Ginkgo Biloba extract composite formulation according to claim 3, characterized in that, comprising: adding an excipient to the Ginkgo Biloba extract, Salvia miltiorrhiza extract, astaxanthin, Astragalus, Coenzyme Q10, and American Ginseng, and processing the mixture to prepare any one of a pill, a capsule, a granule, a tablet, and an oral solution.

9. A use of the Ginkgo Biloba extract composite formulation according to claim 2, characterized in that, comprising: adding an excipient to the Ginkgo Biloba extract, Salvia miltiorrhiza extract, astaxanthin, Astragalus, Coenzyme Q10, and American Ginseng, and processing the mixture to prepare any one of a pill, a capsule, a granule, a tablet, and an oral solution.

10. A use of the Ginkgo Biloba extract composite formulation according to claim 1, characterized in that, comprising: adding an excipient to the Ginkgo Biloba extract, Salvia miltiorrhiza extract, astaxanthin, Astragalus, Coenzyme Q10, and American Ginseng, and processing the mixture to prepare any one of a pill, a capsule, a granule, a tablet, and an oral solution.
